(19) United States
(12) Patent Application Publication
(10) Pub. No.: US 2025/0115848 A1
(43) Pub. Date: Apr. 10, 2025

(54) CULTURE APPARATUS

(71) Applicants: Canon Kabushiki Kaisha, Tokyo (JP); CANON MEDICAL SYSTEMS CORPORATION, Tochigi (JP)

(72) Inventors: Tomoki HANEISHI, Kita-ku (JP); Tomoki FUJITO, Nasushiobara (JP)

(73) Assignees: Canon Kabushiki Kaisha, Tokyo (JP); CANON MEDICAL SYSTEMS CORPORATION, Tochigi (JP)

(21) Appl. No.: 18/906,306

(22) Filed: Oct. 4, 2024

(30) Foreign Application Priority Data
Oct. 6, 2023 (JP) 2023-174552

(51) Int. Cl. C12M 1/00; C12M 1/12

(52) U.S. Cl. CPC C12M 29/00; C12M 23/22; C12M 23/26; C12M 25/01

(57) ABSTRACT

A culture apparatus according to the present embodiment includes a supply unit, a housing, and a holding mechanism. The supply unit supplies a culture vessel with a culture medium for culturing of cells. The housing has the supply unit disposed therein and constitutes a confined space with the culture vessel. The holding mechanism is disposed in the housing and holds the culture vessel. The culture apparatus according to the present embodiment makes it possible to perform cell culturing corresponding to various purposes in the closed condition.

### CROSS-REFERENCE TO RELATED APPLICATIONS

This application is based upon and claims the benefit of priority from Japanese Patent Application No. 2023-174552, filed on Oct. 6, 2023; the entire contents of which are incorporated herein by reference.

### FIELD

Embodiments described herein relate generally to a culture apparatus.

### BACKGROUND

There are two types of cell culturing, namely, open-type culturing and closed-type culturing. The open-type culturing is a commonly-used culturing method in which, usually, culturing is performed using a dish representing a culture vessel, and tasks such as culture medium replacement and passage culture are performed by the worker by opening the lid of the dish. In the open-type culturing, since the space used for cell culturing is basically in the open condition, there is a possibility of bacteria getting mixed in the culture vessel. In the closed-type culturing, although cell culturing can be performed in a closed-type (sealed) culture vessel, the tasks such as culture medium replacement are performed by the worker in the open condition. Hence, after all, there is a risk of bacteria getting mixed. For that reason, there is a demand for a culture apparatus that enables performing culture medium replacement and cell detachment too in a closed condition.

### BRIEF DESCRIPTION OF THE DRAWINGS

FIG. 1 is a perspective view illustrating a configuration of a culture apparatus according to an embodiment;

FIGS. 2 and 3 are sectional side views illustrating a configuration of the culture apparatus according to the present embodiment;

FIG. 4 is a side view of a second housing portion in the culture apparatus according to the present embodiment, and is a diagram for explaining an example of a holding mechanism; and

FIGS. 5A and 5B are diagrams for explaining the problems faced when an attempt is made to hold the culture vessel at the inner diameter of the housing portion.

### DETAILED DESCRIPTION

A culture apparatus according to an embodiment includes a supply unit, a housing, and a holding mechanism. The supply unit supplies a culture vessel with a culture medium for culturing of cells. The housing has the supply unit disposed therein and constitutes a confined space with the culture vessel. The holding mechanism is disposed in the housing and holds the culture vessel. The culture apparatus according to the present embodiment makes it possible to perform cell culturing corresponding to various purposes in the closed condition.

An exemplary embodiment of a culture apparatus is described below in detail with reference to the accompanying drawings. However, the embodiments are not limited to the embodiment described below. Moreover, the details given in a single embodiment are applicable in principle to other embodiments too in an identical manner.

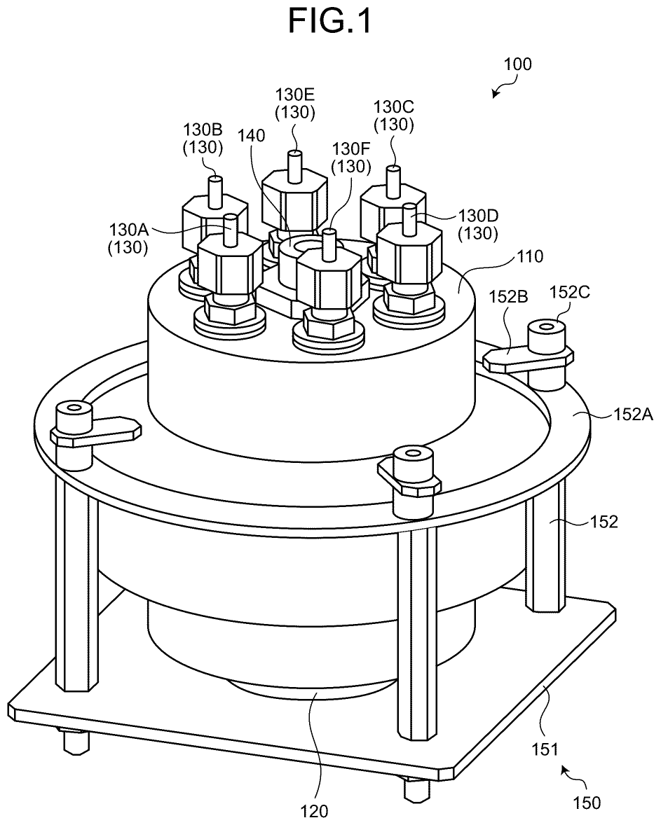

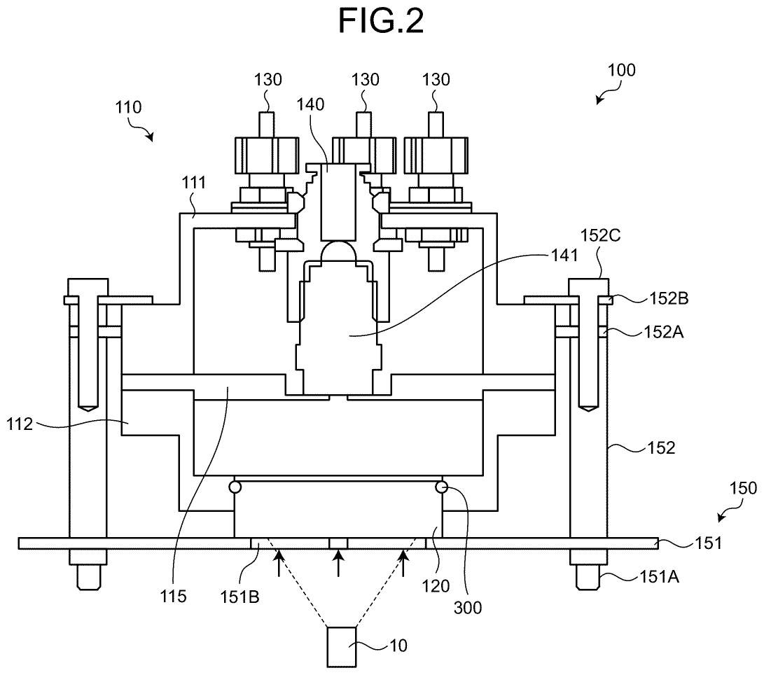

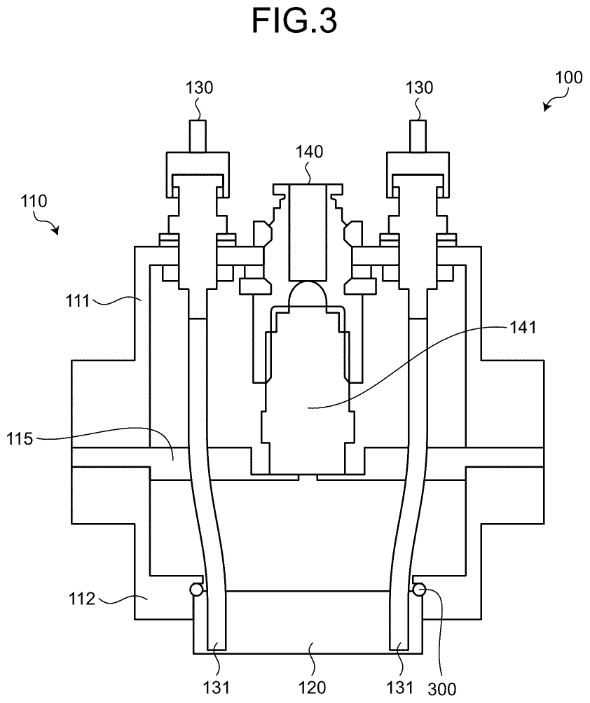

FIG. 1 is a perspective view illustrating a configuration of a culture apparatus 100 according to the present embodiment. FIGS. 2 and 3 are sectional side views illustrating a configuration of the culture apparatus 100 according to the present embodiment. In FIG. 2, tubes 131 illustrated in FIG. 3 are omitted. In FIG. 3, a support member 150 illustrated in FIG. 2 is omitted.

As illustrated in FIG. 1, the culture apparatus 100 according to the present embodiment includes a housing 110, a light shielding plate 115, and a plurality of couplings 130.

The housing 110 has a cylindrical portion in which tubes (explained later) are disposed; and includes a first housing portion 111 and a second housing portion 112 that is disposed in the lower part of the first housing portion 111. The light shielding plate 115 is disposed in between the first housing portion 111 and the second housing portion 112. In the culture apparatus 100 according to the present embodiment, an arbitrary sealing mechanism is disposed in between the first housing portion 111 and the light shielding plate 115 as well as in between the light shielding plate 115 and the second housing portion 112. As a result, the sealing property of the housing 110 is secured.

The first housing portion 111, the light shielding plate 115, and the second housing portion 112 have, for example, screw holes formed thereon in the vertical direction and are screw-clamped together. That is, the first housing portion 111 and the second housing portion 112 support the light shielding plate 115.

In the culture apparatus 100 according to the present embodiment, in order to reduce the infection risk to the worker, tubes are used for enabling culture medium replacement and cell collection in the closed condition.

In the upper part of the first housing portion 111, the plurality of couplings 130 are disposed. As illustrated in FIG. 3, the culture apparatus 100 according to the present embodiment further includes a plurality of tubes 131. Each of the plurality of tubes 131 is disposed inside the housing 110 via one of the plurality of couplings 130.

For example, the plurality of tubes 131 include a tube for cell seeding and culture medium replacement, a tube for waste solution collection, a tube for PBS addition, a tube for EDTA addition, a tube for cell suspension liquid collection, and a spare tube. In the example illustrated in FIG. 1, the plurality of couplings 130 include a coupling 130A that is connected to the tube 131 for cell seeding and culture medium replacement; a coupling 130B that is connected to the tube 131 for waste solution collection; a coupling 130C that is connected to the tube 131 for PBS addition; a coupling 130D that is connected to the tube 131 for EDTA addition; a coupling 130E that is connected to the tube 131 for cell suspension liquid collection; and a coupling 130F that is connected to the spare tube 131.

As illustrated in FIG. 3, inside the housing 110, a plurality of tubes 131 is disposed. As illustrated in FIG. 3, on the light shielding plate 115, openings are formed through which the plurality of tubes 131 are disposed.

As illustrated in FIGS. 2 and 3, in the bottom part of the second housing portion 112, a culture vessel 120 is disposed that represents a dish for culturing cells. The bottom face of the culture vessel 120 has a circular shape. On top of the culture vessel 120, the culture apparatus 100 according to the present embodiment is placed as a lid. Inside the culture vessel 120, the leading ends of the plurality of tubes 131 are disposed.

In the culture apparatus 100 according to the present embodiment, a nozzle is used for performing cell detachment in the closed condition.

As illustrated in FIGS. 1 to 3, the culture apparatus 100 according to the present embodiment further includes a coupling 140. Moreover, as illustrated in FIGS. 2 and 3, the culture apparatus 100 according to the present embodiment further includes a detachment nozzle 141.

The coupling 140 is disposed in the central portion of the upper part of the first housing portion 111. The detachment nozzle 141 is disposed inside the housing 110 and is connected to the coupling 140 in the upper part of the first housing portion 111. In the central portion of the light shielding plate 115, an opening is formed into which the leading end of the detachment nozzle 141 is disposed.

As illustrated in FIG. 1, the culture apparatus 100 and the culture vessel 120 according to the present embodiment are supported by the support member 150.

The support member 150 includes a first-type support member 151 and second-type support members 152. The first-type support member 151 supports the culture vessel 120 that is disposed in the bottom part of the second housing portion 112; and the culture vessel 120 is disposed in the central portion of the first-type support member 151.

For example, the second-type support members 152 are columnar in shape, and the first-type support member 151 has through holes formed thereon for allowing passage of the second-type support member 152. Moreover, in each second-type support member 152, a member 152A for restricting the movement of the housing 110 is disposed with a clearance gap maintained from the housing 110. The inner diameter of the member 152A corresponds to the outer diameter of the housing 110. At the upper end of each second-type support member 152, a member 152B is disposed for pressing the first housing portion 111 from the upper side. From the bottom face of the first-type support member 151 on which the through holes are formed, the worker puts in nuts 151A and screw-clamps the first-type support member 151 to the second-type support members 152. Then, the worker inserts a screw 152C from the upper end of each second-type support member 152 and presses the member 152B, which is disposed at the upper end of the concerned second-type support member 152, with pressure from the upper side. That provides support to the culture apparatus 100 and the culture vessel 120 according to the present embodiment.

The following explanation is given about the role of the light shielding plate 115.

For example, the worker observes the inside of the culture vessel 120 using a camera. As illustrated in FIG. 2, in the central portion of the first-type support member 151, an observation window 151B is provided for enabling the worker to observe the inside of the culture vessel 120 using a camera 10. Herein, the observation window 151B can be in the form of an opening. The light shielding plate 115 that is disposed inside the housing 110 is provided for enabling observation using the camera 10; and, when the culture vessel 120 is illuminated from the side, is used to prevent the detachment nozzle 141 and the tubes 131 from being photographed.

Herein, the plurality of tubes 131 and the detachment nozzle 141 are disposed inside the housing 110; and the housing 110 constitutes a confined space.

The following explanation is given about the role of the plurality of tubes 131 and the detachment nozzle 141.

In the culture apparatus 100 according to the present embodiment, the detachment nozzle 141 is used because no lid is provided for enabling culture medium replacement and cell detachment in the closed condition. In the culture apparatus 100 according to the present embodiment, for example, phosphate-buffered saline (PBS) is sprayed as the liquid droplets from the detachment nozzle 141; and the cells adhered to the bottom of the culture vessel 120 are detached, and the detached cells are sucked in and collected through the tube 131 for cell suspension liquid collection.

More particularly, in the culture apparatus 100 according to the present embodiment, firstly, a process is performed in which cells are seeded in the culture vessel 120 using the tube 131 for cell seeding and culture medium replacement. Then, in the culture apparatus 100 according to the present embodiment, a process is performed in which the culture medium in the culture vessel 120 is replaced using the tube 131 for cell seeding and culture medium replacement and the tube 131 for waste solution collection. Since the cells inside the culture vessel 120 are unclean due to the waste matter such as the culture medium, in the culture apparatus 100 according to the present embodiment, a process is performed in which the cells inside the culture vessel 120 are cleaned by adding PBS as a cleaning fluid using the tube 131 for PBS addition, and then the PBS is collected using the tube 131 for waste solution collection. Subsequently, in the culture apparatus 100 according to the present embodiment, a process is performed in which the adhesion force among the cells and the adhesion force between the cells and the culture vessel 120 is weakened by adding ethylenediaminetetraacetic acid (EDTA) as a liquid solution into the culture vessel 120 using the tube 131 for EDTA addition, and then the EDTA is collected using the tube 131 for waste solution collection. Subsequently, in the culture apparatus 100 according to the present embodiment, a process is performed in which PBS liquid droplets are sprayed into the culture vessel 120 using the detachment nozzle 141, so that the water droplets are applied inside the culture vessel 120 and the cells adhered to the bottom of the culture vessel 120 are detached. Lastly, in the culture apparatus 100 according to the present embodiment, a process is performed in which the cells that have been detached inside the culture vessel 120 are sucked in and collected using the tube 131 for cell suspension liquid collection. At that time, a culture medium is added using the tube 131 for medium replacement, and the added culture medium and the cells are collected using the tube 131 for waste solution collection and the tube 131 for cell suspension liquid collection. All the abovementioned processes are performed in the closed condition.

Meanwhile, the tube 131 for cell seeding and culture medium replacement represents an example of a “supply unit”. The detachment nozzle 141 represents an example of a “discharge unit”.

In the case of configuring a culture apparatus in which culture medium replacement is to be performed in the closed condition, it is possible to think of designing the culture vessel (culture chamber) in accordance with the entire culture apparatus. However, if a culture vessel is to be modified depending on the purpose of culturing, it becomes necessary to design a different apparatus.

In that regard, in order to enable cell culturing corresponding to various purposes in the closed condition, the culture apparatus 100 according to the present embodiment includes a supply unit (the tube 131 for cell seeding and culture medium replacement), the housing 110, and holding mechanisms 300. The supply unit supplies the culture vessel 120 with a culture medium for cell culturing. The housing 110 has the supply unit disposed therein, and constitutes a confined space with the culture vessel 120. The holding mechanisms 300 are disposed in the housing 110 and hold the culture vessel 120.

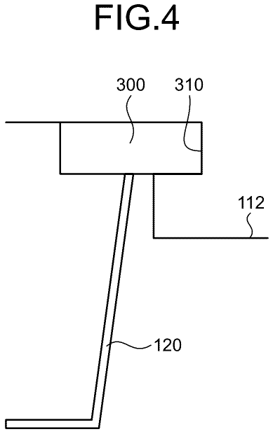

FIG. 4 is a side view of the second housing portion 112 in the culture apparatus 100 according to the present embodiment, and is a diagram for explaining an example of the holding mechanism 300.

Each holding mechanism 300 includes an elastic member. For example, the elastic member is an O-ring. More particularly, the elastic member is an O-ring made of silicon rubber. With reference to FIG. 4, as a result of the contact made by the elastic member with the upper portion of a sidewall of the culture vessel 120, the holding mechanism 300 holds the culture vessel 120.

As illustrated in FIG. 4, in the second housing portion 112 of the housing 110, a groove 310 is formed in the portion that comes in contact with the upper portion of a sidewall of the culture vessel 120. With reference to FIG. 4, the groove 310 is formed on the entrance-side end face of the second housing portion 112. In the holding mechanism 300, the elastic member gets sandwiched in the groove 310 formed on the entrance-side end face of the second housing portion 112 of the housing 110.

Each holding mechanism 300 supports the bottom face of the culture vessel 120 from the underside and also causes the housing 110 to make contact with the upper end of the culture vessel 120 so that the housing 110 is pressed from the upper portion thereof. As a result, the holding mechanisms 300 constitute a confined space and hold the culture vessel 120. Regarding incorporating the culture vessel 120 into the culture apparatus 100 according to the present embodiment, the explanation is given with reference to FIGS. 2 and 4.

Firstly, the worker places the culture vessel 120 in the central portion of the first-type support member 151 and ensures that the housing 110 makes contact with the upper end of the culture vessel 120. Then, after supporting the bottom face of the culture vessel 120 from the first-type support member 151, the worker passes the second-type support members 152 through the through holes formed on the first-type support member 151; puts in the nuts 151A from the bottom face of the first-type support member 151 having the through holes formed thereon; and screw-clamps the first-type support member 151 to the second-type support members 152. Subsequently, the worker inserts the screws 152C from the upper end of the second-type support members 152 and presses the screws 152C, which are provided at the upper end of the second-type support members 152, from the upper side with pressure. At that time, the upper portion of the sidewalls of the culture vessel 120 run into the elastic members of the holding mechanisms 300 that are disposed on the entrance-side end faces of the second housing portion 112 of the housing 110; and the elastic members collapse due to the upper portion of the sidewalls of the culture vessel 120. As a result, the holding mechanisms 300 constitute a confined space and hold the culture vessel 120.

In the culture apparatus 100 according to the present embodiment, the configuration is such that the elastic members of the holding mechanisms 300 collapse due to the upper portion of the sidewalls of the culture vessel 120. That enables dealing with the difference in the shape of the culture vessel 120 depending on the manufacturer. That is, in the culture apparatus 100 according to the present embodiment, any commercially available culture vessel 120 having a different shape can be used.

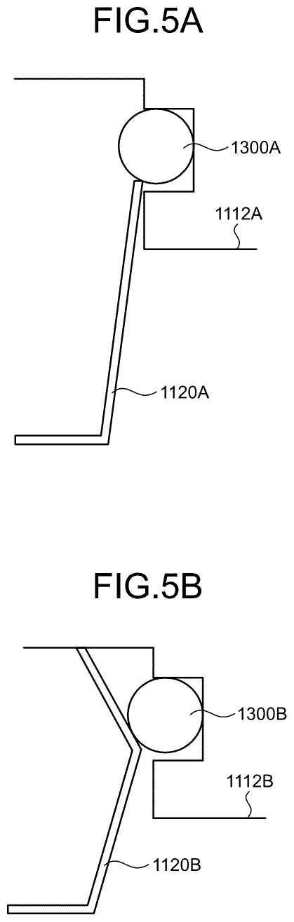

FIGS. 5A and 5B are diagrams for explaining the problems faced when an attempt is made to hold the culture vessel at the inner diameter of the housing portion.

For example, in FIG. 5A is illustrated a case of using a culture vessel 1120A manufactured by the company A. In FIG. 5A, when the elastic member of a holding mechanism 1300A is provided on the inner diameter face of a housing portion 1112A, the upper portion of a sidewall of the housing portion 1112A cannot run into the elastic member. As a result, the holding mechanism 1300A neither can constitute a confined space nor can hold the culture vessel 1120A.

For example, in FIG. 5B is illustrated a case of using a culture vessel 1120B manufactured by the company B. In an identical manner to FIG. 5A, in FIG. 5B too, when the elastic member of a holding mechanism 1300B is provided on the inner diameter face of a housing portion 1112B, the elastic member does not collapse sufficiently. As a result, the holding mechanism 1300B neither can constitute a confined space nor can hold the culture vessel 1120B.

In contrast, in the culture apparatus 100 according to the present embodiment, the elastic members of the holding mechanisms 300 are provided on the entrance-side end faces of the second housing portion 112. Hence, regardless of whether the culture vessel 1120A manufactured by the company A is used or the culture vessel 1120B manufactured by the company B is used, the upper portion of the sidewalls of the concerned culture vessel run into the elastic members of the holding mechanisms 300 provided on the entrance-side end faces of the second housing portion 112, and the elastic members collapse due to the upper end of the sidewalls of that culture vessel. As a result, the holding mechanisms 300 can constitute a confined space as well as can hold the culture vessel 1120A manufactured by the company A or the culture vessel 1120B manufactured by the company B.

Meanwhile, since the elastic members of the holding mechanisms 300 are made of rubber (silicon rubber), the culture vessel 120 becomes detachably attachable to the housing 110.

As explained above, in the culture apparatus 100 according to the present embodiment, the supply unit (the tube 131 for cell seeding and culture medium replacement) supplies the culture vessel 120 with a culture medium for cell culturing, and the discharge unit (the detachment nozzle 141) discharges the liquid droplets for detaching the cells placed in the culture vessel 120. Moreover, in the culture apparatus 100 according to the present embodiment, the housing 110 has the supply unit and the discharge unit disposed therein, and constitutes a confined space with the culture vessel 120; and the holding mechanisms 300 are disposed in the housing 110 for holding the culture vessel 120. Furthermore, the culture vessel 120 is detachably attachable to the housing 110. Hence, the culture apparatus 100 according to the present embodiment can deal with the differences in the shape of the culture vessel 120 depending on the manufacturer, and makes it possible to perform cell culturing corresponding to various purposes in the closed condition. Meanwhile, in the culture apparatus 100 according to the present embodiment, since cell medium replacement can be performed in the closed condition, the discharge unit (the detachment nozzle 141) need not be disposed.

According to at least one embodiment described above, it becomes possible to perform cell culturing corresponding to various purposes in the closed condition.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A culture apparatus comprising:
a supply unit that supplies a culture vessel with a culture medium for culturing of cells;
a housing that has the supply unit disposed therein and that constitutes a confined space with the culture vessel; and
a holding mechanism that is disposed in the housing and that holds the culture vessel.

2. The culture apparatus according to claim 1, wherein, the holding mechanism supports bottom face of the culture vessel from underside and also causes the housing to make contact with upper end of the culture vessel so that the housing is pressed from the upper portion thereof, so as to constitute the confined space and hold the culture vessel.

3. The culture apparatus according to claim 2, further comprising a support member that supports bottom face of the culture vessel from underside and that presses the housing from upper side, wherein
when the housing is pressed by the support member, the holding mechanism holds the culture vessel.

4. The culture apparatus according to claim 3, wherein the support member includes
a first-type support member that supports bottom face of the culture vessel from underside,
a second-type support member that is linked to the first-type support member, and
a member that is disposed at upper end of the second-type support member and that presses the housing from upper side.

5. The culture apparatus according to claim 4, wherein the first-type support member has an observation window provided therein for enabling observation of inside of the culture vessel.

6. The culture apparatus according to claim 1, wherein the holding mechanism includes an elastic member and holds the culture vessel when the elastic member makes contact with upper portion of a sidewall of the culture vessel.

7. The culture apparatus according to claim 6, wherein the elastic member is sandwiched in a groove formed in the housing.

8. The culture apparatus according to claim 1, further comprising a discharge unit that discharges liquid droplets for detaching the cells placed in the culture vessel.

9. The culture apparatus according to claim 8, wherein the supply unit and the discharge unit are disposed inside the housing.

10. A culture apparatus comprising:
a supply unit that supplies a culture vessel with a culture medium for culturing of cells; and
a housing that has the supply unit disposed therein and that constitutes a confined space with the culture vessel, wherein
the culture vessel is detachably attachable to the housing.
